(19) United States
(12) Patent Application Publication
(10) Pub. No.: US 2010/0179632 A1
(43) Pub. Date: Jul. 15, 2010

(54) Robotic Fenestration Device Having Impedance Measurement

(75) Inventors: Walter Bruszewski, Guerneville, CA (US); James Machek, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/352,254

(22) Filed: Jan. 12, 2009

(51) Int. Cl. A61F 2/84; A61B 5/053; A61B 19/00

(52) U.S. Cl. 623/1.11; 600/547; 606/130

(57) ABSTRACT

A method and system for real-time continuous impedance monitoring along the surface of a graft implanted within a main vessel to aid in optimally positioning an electrode at a branch vessel ostium. Due to the conductivity differences among various kinds of solid tissue and blood, a fenestration catheter system uses impedance monitoring as a tool to detect the location of branch ostia through graft cloth. Such information enables the fenestration electrode to be properly positioned for creation of a fenestration in the graft cloth in situ. In addition, the fenestration catheter system may utilize impedance information to avoid contact between the electrode and metal stent structures used to anchor the graft during an in situ fenestration procedure. The fenestration catheter system includes a catheter shaft, an electrode, one or more reference or indifferent electrodes, an impedance analyzer, a power source and an electrode position reference to record impedance measurements in relation to position.

### FIELD OF THE INVENTION

The present invention relates generally to methods and systems for providing perfusion to branch vessels when a graft is implanted in a body lumen for the treatment of vascular disease.

### BACKGROUND

Prostheses for implantation in blood vessels or other similar organs of the living body are, in general, well known in the medical art. For example, prosthetic vascular grafts constructed of biocompatible materials, such as Dacron or expanded porous polytetrafluoroethylene (ePTFE) tubing, have been employed to replace or bypass damaged or occluded natural blood vessels. In general, endovascular grafts typically include a graft anchoring component that operates to hold the tubular graft in its intended position within the blood vessel. Commonly, for grafts which are delivered using a minimally invasive approach (stent grafts) the graft anchoring component is one or more radially compressible stents that are radially expanded in situ to anchor the tubular graft to the wall of a blood vessel or anatomical conduit. Thus, endovascular grafts are typically held in place by mechanical engagement and friction due to the opposition forces provided by the expanded stents.

In general, rather than performing an open surgical procedure to implant a bypass tubular graft that may be traumatic and invasive, stent grafts are preferably deployed through a less invasive intraluminal delivery. More particularly, a lumen of the vasculature is accessed at a convenient entry point, and the stent graft is routed through the vasculature to the site where the prosthesis is to be deployed. Intraluminal deployment is typically effected using a delivery catheter with coaxial inner and outer tubes arranged to move axially relative to each other. For example, a self-expanding stent graft may be radially compressed and disposed within the distal end of an outer catheter tube distal of a stop fixed to the inner member. The end of the catheter containing the stent graft is then maneuvered, typically routed though a body lumen until the end of the catheter and the stent graft is positioned at or adjacent to the intended treatment site. The inner member is then held stationary while the outer tube of the delivery catheter is withdrawn. The stop fixed to the inner member prevents the stent graft from being withdrawn with the sheath. As the sheath is withdrawn, the end of the stent graft is gradually exposed and is released from the confines of the sheath and radially self-expands so that at least a portion of it contacts and substantially conforms to a portion of the surrounding interior of the lumen, e.g., the blood vessel wall or anatomical conduit. The sheath is withdrawn to release the stent graft fully.

Grafting procedures are also known for treating aneurysms. Aneurysms result from weak, thinned blood vessel walls that “balloon” or expand due to aging, disease and/or blood pressure in the vessel. Consequently, aneurysmal vessels have a potential to rupture, causing internal bleeding and potentially life threatening conditions. Grafts are often used to isolate aneurysms or other blood vessel abnormalities from normal blood pressure, reducing pressure on the weakened vessel wall and reducing the chance of vessel rupture. As such, a tubular endovascular graft may be placed within the aneurysmal blood vessel to create a new flow path and an artificial flow conduit through the aneurysm, thereby reducing if not nearly eliminating the exertion of blood pressure on the aneurysm.

While aneurysms can occur in any blood vessel, most occur in the aorta and peripheral arteries. Depending on the region of the aorta involved, the aneurysm may extend into areas of bifurcation or segments of the aorta from which smaller “branch” arteries extend. Various types of aortic aneurysms may be classified on the basis of the region of aneurysmic involvement. For example, thoracic aortic aneurysms include aneurysms present in the ascending thoracic aorta, the aortic arch, and branch arteries that emanate therefrom, such as subclavian arteries. Thoracoabdominal aortic aneurysm include aneurysms present in the descending thoracic aorta and branch arteries that emanate therefrom, such as thoracic intercostal arteries and/or the suprarenal abdominal aorta and branch arteries that emanate therefrom, such as superior mesenteric, celiac and/or intercostal arteries. Lastly, abdominal aortic aneurysms include aneurysms present in the pararenal aorta and the branch arteries that emanate therefrom, such as the renal arteries.

Unfortunately, not all patients diagnosed with aortic aneurysms are presently considered to be candidates for endovascular grafting. This is largely due to the fact that the anatomy of branch vessels is variable amongst patients, and most of the endovascular grafting systems of the prior art are not designed for use in regions of the aorta from which branches extend. The deployment of endovascular grafts within regions of the aorta from which branch arteries extend, present additional technical challenges because in those cases the endovascular graft must be designed, implanted, and maintained in a manner which does not impair the flow of blood into the branch arteries.

To provide perfusion to branches, a stent graft having a preconfigured fenestration or opening in a side wall thereof is utilized. The stent graft is positioned so that fenestration is aligned with the ostium of the branch vessel after deployment of the stent graft. In use, the proximal end of the graft having one or more side openings is securely anchored in place, and the fenestrations or openings are configured and deployed to provide blood flow into the branches. In some cases, another stent graft, often referred to as a branch graft, may then be deployed through the fenestration into the branch vessel to seal or stabilize a path for blood flow to the branch vessel. One challenge that exists in such a procedure is how to accurately position the fenestration of the main stent graft in relation to the branch vessel. If the stent graft is deployed in a main vessel and the position of a fenestration ends up offset from the branch vessel which it is intended to feed, it may be difficult to deploy guidewires and catheters from the stent graft into the branch vessel to enable delivery and/or correct positioning of the branch vessel stent graft. Poor positioning of a branch vessel stent graft may cause the branch graft to kinks to such an extent that blood flow will be restricted or will not occur therethrough. Thus, there remains a need in the art for the development of new endovascular systems and methods to provide perfusion to branch vessels from an implanted tubular stent graft in a main vessel.

### SUMMARY OF THE INVENTION

A system that facilitates fenestration of an endoluminal graft in situ is disclosed. The system includes a tubular shaft defining at least one lumen and at least one lead extending through the at least one lumen of the tubular shaft. A radiofrequency (RF) electrode capable of creating a fenestration in a wall of the endoluminal graft is attached to a distal end of the lead. A power source is electrically connected to the RF electrode for generating an AC current. An impedance analyzer is electrically connected to the RF electrode and to a common reference electrode. The impedance analyzer continuously measures the impedance between the RF electrode and the common reference electrode, wherein analysis of the impedance measurements reveal an optimal location for creating a fenestration in a wall of the endoluminal graft.

The system may also include a robotic steering interface for deflecting and rotating the system in all direction, and a display for producing a virtual 3D real time representation of the position and orientation of the system within the vasculature. The display may overlay a virtual impedance map of impedance measurements onto the virtual 3D real time representation.

### BRIEF DESCRIPTION OF DRAWINGS

The foregoing and other features and advantages of embodiments in accordance herewith will be apparent from the following description as illustrated in the accompanying drawings. The drawings are not to scale.

FIG. 1 is a schematic diagram of a fenestration catheter system according to an embodiment hereof.

FIGS. 2-7 are schematic diagrams of a fenestration catheter system being utilized to create a fenestration in situ within a wall of an implanted graft to perfuse a branch vessel.

FIG. 8 is a schematic representation of relative impedance zones encountered as a fenestration catheter system according to an embodiment hereof is tracked along an interior surface of graft material.

FIG. 9 is a schematic diagram of a fenestration catheter system according to another embodiment hereof.

### DETAILED DESCRIPTION

Specific embodiments are now described with reference to the figures, wherein like reference numbers indicate identical or functionally similar elements. When the terms “distal” and “proximal” are used in relation to a delivery system or a catheter system in the following description with respect to a position or direction relative to the treating clinician. “Distal” or “distally” are a position distant from or in a direction away from the clinician. “Proximal” and “proximally” are a position near or in a direction toward the clinician. However, when referring to the graft (whether in the delivery system or already implanted) the proximal end refers to the end of the graft material nearest the heart by way of blood flow path, while distal is the end farthest from the heart by way of blood flow path.

The following detailed description is merely exemplary in nature. Although the description herein is in the context of treatment of blood vessels such as the aortic, carotid, and renal arteries, embodiments according to the present invention may also be used in any other body passageways where deemed useful.

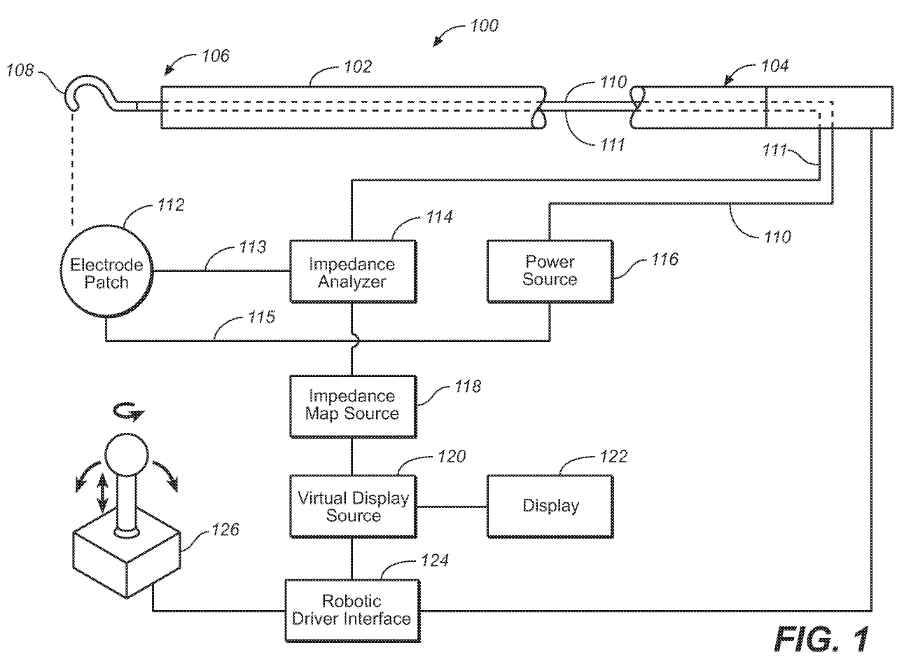

With reference to FIG. 1, there is shown a schematic diagram of a system for continuously monitoring impedance along a surface of an implanted graft to reveal the precise location of a branch vessel ostium and then create a fenestration in situ at the ostium for perfusion of the branch vessel. Fenestration catheter system 100 includes a catheter shaft 102, a first electrode 108 disposed on a distal portion of the device, a second electrode (patch) 112 disposed separately from the first electrode 108, a power source 116, and an impedance analyzer 114 for connection to the first and second electrodes 108, 112. As the first electrode 108 of the catheter system 100 is advanced along a surface of an implanted graft, an electric current is supplied between the first and second electrodes 108, 112. Resistance between the first and second electrodes 108, 112 is measured and converted arithmetically to a calculated bioelectric impedance via impedance analyzer 114 to measure impedance between the two electrodes. Due to the known conductivity differences among various kinds of solid tissue and blood, the measured/calculated impedance allows a clinician to determine the location of branch ostia through the graft cloth as the position of the electrode is recorded relative to the measured/calculated impedance value and electrode position reference locations (which coarsely could be established by a clinician making grease pen marks on a radiographic image display screen and which could be more precisely established and recorded automatically by computer recorded and activated electrode position adjustment) as it moves along the inner surface of the stent graft near and over a branch ostium. With this information, the clinician can identify the location on the inside of the stent graft at which to create a fenestration in the graft cloth in situ to perfuse the branch vessel.

Catheter shaft 102 is an elongate flexible tubular shaft defining at least one lumen extending from a proximal end 104 to a distal end 106 thereof. Proximal end 104 of catheter shaft 102 extends out of the patient and is available to be manipulated by a clinician, and distal end 106 of catheter shaft 102 is positionable at a target location within the vasculature. The lumen of catheter shaft 102 may have any suitable cross-section, including a circular cross-section or an elliptical cross-section. It is desirable for the lumen to be as small as possible to minimize the outer diameter of catheter shaft 102, thus minimizing the crossing profile of fenestration catheter system 100 so that it fits within relatively small vessels.

Catheter shaft 102 may have any suitable working length, for example, 50 cm-200 cm, suitable to extend to a target location within the vasculature. Catheter shaft 102 can be of any suitable construction and made of any suitable material, such as an extruded shaft formed of any suitable flexible polymeric material. Non-exhaustive examples of polymeric materials for catheter shaft 102 are HDPE, PEBAX, polyethylene terephalate (PET), nylon, silicone, polyethylene, LDPE, HMWPE, polyurethane, or combinations of any of these, either blended or co-extruded. In one embodiment, a proximal portion of the catheter may be formed of a metallic material, such as stainless steel or nitinol, or as a composite having a reinforcement material incorporated within a polymeric body in order to enhance strength, flexibility, and/or toughness. Suitable reinforcement layers include braiding, wire mesh layers, embedded axial wires, embedded helical or circumferential wires, and the like. In an embodiment, the proximal portion of the catheter may in some instances be formed from a reinforced polymeric tube, for example, as shown and described in U.S. Pat. No. 5,827,242 to Follmer et al., which is incorporated by reference herein in its entirety.

First electrode 108 can be utilized to create a fenestration in graft cloth and is disposed at a distal portion of fenestration catheter system 100. Electrode 108 may be a radiofrequency (RF) electrode, and may have any suitable configuration capable of creating a fenestration in the sidewall of a graft. For example, electrode 108 may be a RF dome or ring electrode, including but not limited to those shown or described in U.S. patent application Ser. No. 11/939,106 to Bruszewski et al. filed Nov. 13, 2007, U.S. Patent Appl. Pub. No. US 2007/0208256 to Marilla, or WO 2007/082343 to Mclachlan et al., all of which are incorporated by reference herein in their entirety. In one embodiment, as shown in FIG. 1, electrode 108 has at least a partial ring (or hook) shape when it has been extended out of catheter shaft 102. Other suitable configurations for electrode 108 include a dome-shaped electrode, a straight or puncture-type electrode, a coiled electrode, a flat or disc-type electrode, or any other configuration suitable for creating a fenestration in the graft cloth.

In one embodiment, electrode 108 can be made from a shape memory material to be self-expanding, meaning it has a mechanical memory to return to a preset unconstrained expanded configuration after having been deformed. Mechanical memory may be imparted to electrode 108 by thermal treatment to achieve a spring temper in stainless steel, for example, or to set a shape memory in a susceptible metal alloy, such as nitinol. In a delivery configuration, catheter shaft 102 surrounds and mechanically deforms electrode 108 into a straightened or compressed configuration to minimize the delivery profile of system 100, which eases advancement of system 100 through the vasculature to the treatment site within a body vessel. When it is desired to deploy electrode 108, catheter shaft 102 and electrode 108 may be moved relative to each other such that electrode 108 is released from catheter shaft 102 and allowed to assume its preset (expanded) configuration. To cause the relative motion between catheter shaft 102 and electrode 108, electrode 108 may be distally advanced while catheter shaft 102 is held in place so that electrode 108 is essentially pushed out of the exit port of catheter shaft 102, or catheter shaft 102 may be retracted in a proximal direction while electrode 108 is held in place so that electrode 108 is essentially exposed, or a combination of these movements may be employed. Once electrode 108 exits catheter shaft 102, electrode 108 elastically assumes its expanded configuration.

In one embodiment, electrode 108 can be coated to prevent grounding or errant conduction of electricity or electric fields or currents away from the wire or electrodes. Such coatings may be of a non-conductive ceramic, polyimide Kapton, hi-temp parylene, or other heat resistant dielectric material which can form a coating. Coating thicknesses of approximately 0.001 inches have been found to be sufficient. A source for parylene coatings useful in embodiments hereof is available from Specialty Coating Systems of Indianapolis, Ind.

Fenestration catheter system 100 also includes second electrode 112, which is a common or indifferent reference electrode that is separately disposed from first electrode 108. Second electrode 112 may be positionable outside the patient's body, viz., a skin patch electrode. Alternatively, second electrode may be disposed in a separate intravascular device and be in electrical contact with the patient's tissue at an in situ location spaced apart from first electrode 108. For example, a guidewire could be used with an electrode disposed thereon and placed within the target branch vessel to serve as second electrode 112.

An electrically conductive lead 110 extends through the lumen of catheter shaft 102 and communicates with electrode 108. Lead 110 extends outside of the body to power source 116. In addition, an electrically conductive lead 115 communicates with electrode 112. Lead 115 extends outside of the body to power source 116. Power source 116, in conjunction with leads 110, 115 and electrodes 108, 112 form a complete electrical circuit that includes a portion of a patient's body extending between electrodes 108, 112.

Similarly, an electrically conductive lead 111 extends through the lumen of catheter shaft 102 and communicates with electrode 108. Lead 111 extends outside of the body to impedance analyzer 114. In addition, an electrically conductive lead 113 communicates with electrode 112. Lead 113 extends outside of the body to impedance analyzer 114. Impedance analyzer 114, in conjunction with leads 111, 113 and electrodes 108, 112 form a complete electrical circuit that includes a portion of a patient's body extending between electrodes 108, 112.

Electrode 108 is fixedly attached to the distal ends of leads 110, 111 by any suitable means. For example, electrode 108 may be attached via welding, such as by resistance welding, friction welding, laser welding or another form of welding such that no additional materials are used to connect electrode 108 and leads 110, 111. Alternatively, electrode 108 and leads 110, 111 can be connected by soldering, by the use of an electrically conductive adhesive, by the addition of a connecting element there between, or by another mechanical method. In an embodiment, the distal ends of leads 110, 111 may extend into one or more bores (not shown) of electrode 108 and be fixedly attached thereto. In one embodiment, leads 110 and 111 may be connected to a proximal end of a single conducting element (not shown) which is integral with electrode 108. The single conducting element may be formed from platinum, stainless steel, or nickel-titanium (nitinol) wire.

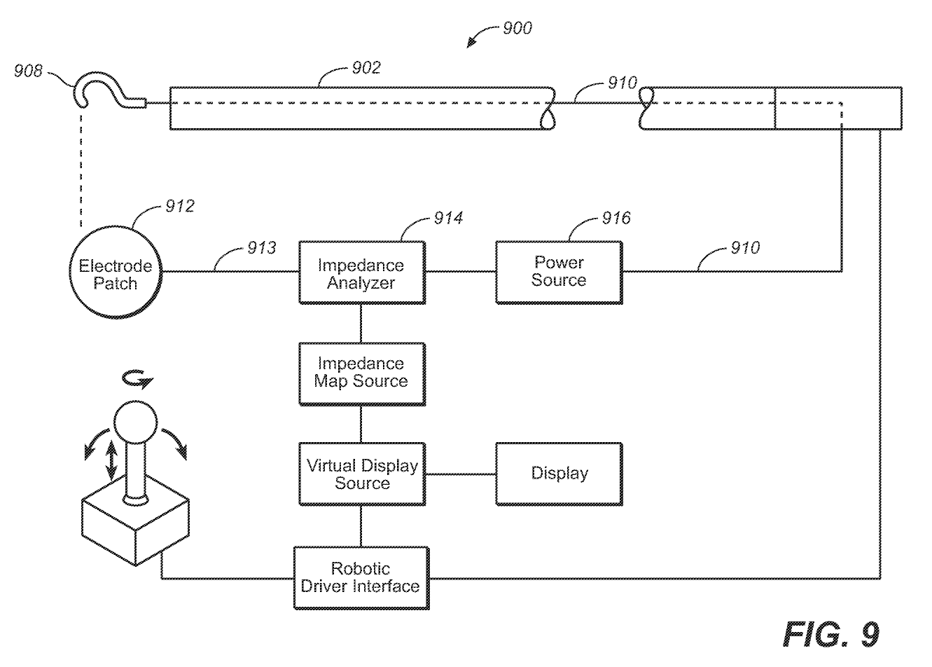

Although fenestration catheter system 100 is described above with reference to FIG. 1 with two completed electrical circuits, i.e., one complete electrical circuit including impedance analyzer 114 and the other complete electrical circuit including power source 116, other configurations are possible. For example, a fenestration catheter system 900 is shown in FIG. 9 with only one complete electrical circuit. In this embodiment, power source 916 and impedance analyzer 914 are connected together externally or they may be combined in an integral device. An electrically conductive lead 910 extends through the lumen of catheter shaft 902 and communicates with electrode 908. A proximal length of lead 910 extends outside of the body to be coupled with power source 916. In addition, an electrically conductive lead 913 connects electrode 912 and impedance analyzer 914. Power source 916 and impedance analyzer 914, in conjunction with leads 910, 913 and electrodes 908, 912 form a complete electrical circuit that includes a portion of a patient's body extending between electrodes 908, 912. Fenestration catheter system 900 includes the same essential functional components and performs in the same manner as fenestration catheter system 100.

Referring back to FIG. 1, fenestration catheter system 100 includes power source 116 that generates a harmless AC electric current through lead 110 to allow for the measurement of impedance, as will be described in further detail below. The current is an alternating current and the selected alternating frequency may be in the range of 1 kHz to 500 kHz or other suitable frequencies known to those of skill in the art of bioelectric impedance. For example, a current of 2 microamperes at 50 KHz may be used. In addition, power source 116 may generate a current sufficient to cut through and create a fenestration in graft cloth. The current may be any current having a power level and frequency that cuts through graft material. In one embodiment, power source 116 may be a RF power generator manufactured by Pfizer Valley Lab of Boulder, Colo., which is capable of providing a high output power between 100 W-200 W. As shown in FIG. 1, power source 116 may be a single device capable of producing a current sufficient to allow for impedance measurement and a current sufficient to create a fenestration in graft cloth, or may be two separate devices (not shown).

Power source 116 and impedance analyzer 114 work together in order to continuously measure impedance in real time along the inner surface (wall) of a previously implanted graft. Real-time impedance measurement will aid in positioning electrode 108 optimally with respect to the surface of the graft cloth, aligned with the ostium of a target branch vessel such that a fenestration may be created via electrode 108 that will perfuse the target branch vessel. In practice, as catheter system 100 is advanced along a surface of an implanted graft, power source 116 generates the AC current through leads 110, 115 and through the patient's tissue between electrodes 108, 112. While current is flowing, impedance analyzer 114 measures a corresponding resistance between the first and second electrodes 108, 112 via leads 111, 113. Impedance analyzer 114 then arithmetically converts the resistance to an impedance measurement. Impedance analyzer 114 may include logic resources, such as a microprocessor, and/or memory resources, such as a RAM or DRAM chip, configured to analyze, store and display bioelectric information derived from electrodes 108, 112. For example, impedance analyzer 114 may include a voltage-current converting circuit, an amplifying circuit, an AID converting circuit, and an impedance arithmetic operation section. In one embodiment, impedance analyzer 114 may be a Medtronic EP RF ablation power supply, such as the ATAKR I RF Generator or ATAKR II® RF Generator, manufactured by Medtronic, Inc. of Minneapolis, Minn., which both measure impedance in real time using the ablation power circuit.

Due to the conductivity differences among various kinds of solid tissue and blood, the measured/calculated impedance allows a clinician to determine the location of branch ostia through graft cloth. More particularly, experimental observations indicate that the impedance or conductivity of blood alone is approximately 50 ohms. However, solid tissue such as the vessel wall will have higher impedance values in the range of 100-200 ohms. Thus, as impedance values are continuously monitored along the inner surface of the graft cloth, graft cloth having solid tissue there behind will have relatively higher impedance values than that associated with graft cloth having only blood there behind. Accordingly, relatively lower impedance values indicate the location of the branch ostium and thus impedance analyzer 114 is able to reveal when the electrode 108 of the fenestration catheter system 100 is properly aligned and/or positioned with respect to the ostium of a target branch vessel. Accordingly, a clinician may use the continuously measured impedance values to determine when electrode 108 is aligned at the proper location to create a fenestration in the graft cloth in situ to perfuse the branch vessel. The clinician may then manually activate power source 116 to generate current sufficient to cut through graft material to create a fenestration in the graft wall thereby permitting blood flow to the branch vessel.

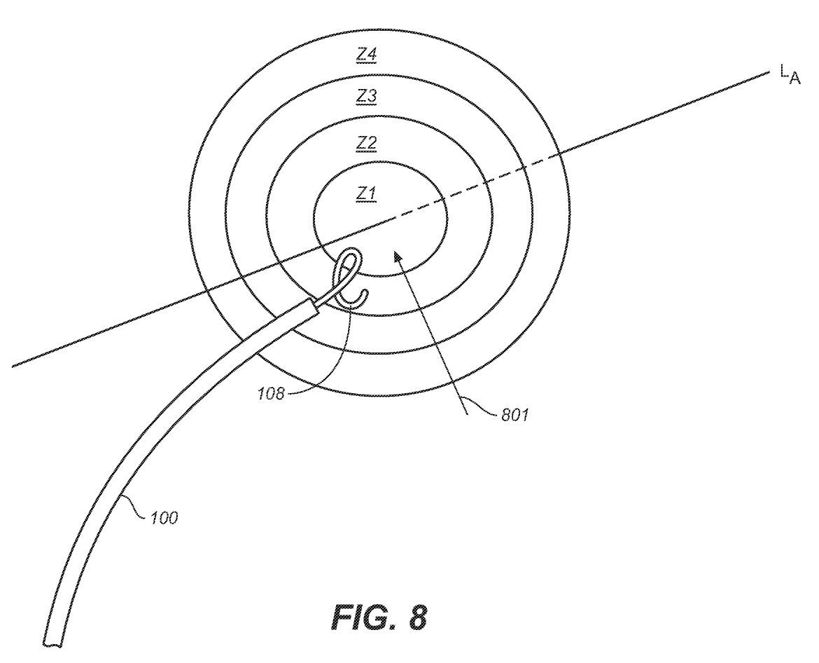

FIG. 8 is a schematic representation of relative impedance “zones” that may be encountered by fenestration catheter system 100 as electrode 108 is tracked along an interior surface of graft material toward an ostium of a target branch vessel. In FIG. 8, zone one (labeled Z1) corresponds to graft material covering the ostium of a target branch vessel having a center or longitudinal axis LA, and zones two, three, and four (labeled Z2, Z3, Z4, respectively) correspond to graft material covering vessel wall. As the electrode 108 of fenestration catheter system 100 approaches Z1, as indicated by directional arrow 801, the measured impedance values will steadily decrease due to the proximity of the ostium of a target branch vessel and the absence of solid tissue there behind. When positioned over Z1, fenestration catheter system 100 will measure impedance of graft cloth having only blood there behind. Thus, the lowest impedance values will occur when electrode 108 is aligned and positioned over Z1. For example, Z1 may measure impedance values in the range of 1,000 ohms-2,000 ohms. When positioned over Z2, Z3, and Z4, fenestration catheter system 100 will measure impedance of graft cloth having vessel wall (tissue) there behind, the vessel wall having a particular proximity to the target ostium. For example, at Z2 impedance values may measure in the range of 2,000 ohms-3,000 ohms; at Z3 impedance values may measure in the range of 3,000 ohms-4,000 ohms; and at Z4 impedance values may measure in the range of 4,000 ohms-5,000 ohms.

It is to be understood that impedance analyzer 114 may be programmed to perform additional functions. In one embodiment, impedance analyzer 114 may be programmed to activate power source 116 to automatically and continuously generate AC current that enables detection of impedance along a surface of an implanted graft. Impedance analyzer 114 may then automatically analyze the various impedance measurements described above to automatically determine when the electrode 108 of the fenestration catheter system 100 is properly aligned with the ostium of a target branch vessel due to the relative impedance measurements. Thus, impedance analyzer 114 may automatically determine the ostium position. Once the target ostium is located, a clinician may then manually activate power source 116 to generate current sufficient to cut through graft material so that electrode 108 will create a fenestration in the graft cloth.

Fenestration catheter system 100 is robotically steerable via robotic driver interface 124 and robotic control 126. Robotic driver interface 124 is a mechanical interface that allows fenestration catheter system 100 to be steered, deflected, and rotated in all directions. As shown in FIG. 1, robotic driver interface 124 is connected to a display 122 via a virtual display source 120. As known in the art, virtual display source 120 provides a virtual 3D real time representation of the position and orientation of fenestration catheter system 100 within the vasculature. In addition to displaying a real time image of the distal end of the fenestration catheter system 100, display 122 also produces a virtual impedance map of the electrical impedance measurements. The virtual impedance map may be overlaid with the anatomical real time image. As shown in FIG. 1, impedance analyzer 114 is connected to display 122 via an impedance map source 118 and virtual display source 120. As impedance analyzer 114 continuously receives signals indicative of impedance, these values are fed into the impedance map source 118 to produce a virtual impedance map of the ostia of target branch vessels. The virtual impedance map is then fed into the virtual display source 120, which may overlay the impedance map onto the 3D real time representation of the position and orientation of fenestration catheter device 100 received from robotic driver interface 124. Display 122 may comprise one or more suitable devices for generating a display and/or maintaining a record of the received signals, and may be incorporated into impedance analyzer 114, RF power source 116, or may be an independent stand alone monitor. In addition or in the alternative to a virtual impedance map of the electrical impedance measurements, a digital readout of the electrical impedance measurements may be obtained. The robotic driver interface 124 may be a Sensei Robotic Catheter System manufactured by Hansen Medical, Inc. of Mountain View, Calif., which may be implemented to include a virtual display of the target tissue anatomy and its electrode activity along with an overlaid virtual representation of a RF ablation system.

In addition to positioning fenestration catheter system 100 at an ostium of a target branch vessel as explained above, impedance analyzer 114 may also be utilized to reveal an optimal fenestration position in which the electrode 108 of the fenestration catheter system 100 is in full contact with cloth and is not in contact with a stent strut. As known in the art, grafts typically include a radially compressible annular support structure or stent attached to a surface of the graft which when released, bias the graft prosthesis into a conforming fixed engagement with an interior surface of the vessel. Examples of such annular support structures are described, for example, in U.S. Pat. No. 5,713,917 to Leonhardt et al. and U.S. Pat. No. 5,824,041 to Lenker et al., which are incorporated by reference herein in their entirety. When used in an aneurysm exclusion device, the stents must have sufficient radial spring force and flexibility to conformingly engage the prosthesis with the body lumen inner wall, to avoid excessive leakage and prevent pressurization of the aneurysm, i.e., to provide a leak-resistant seal. Although some leakage of blood or other body fluid may occur into the aneurysm isolated by the graft prosthesis, an optimal seal will reduce the chances of aneurysm pressurization and resulting rupture. During fenestration of graft cloth, it is important to ensure that the electrode does not touch a stent strut and that the electrode is optimally positioned on the graft cloth because contact with a stent strut may result in cutting the strut and/or an incomplete cut through the graft.

Due to the conductivity differences between graft material and metallic strut materials, the measured/calculated impedance allows a clinician to determine when electrode 108 is in contact with a stent strut. More particularly, when compared to the impedance of graft material alone, experimental observations suggest that the impedance drops when electrode 108 is in contact with a stent strut. Thus, as impedance values are continuously monitored along the graft, relatively lower impedance values indicate that fenestration catheter system 100 is in contact with a strut and that a fenestration should not be created. For example, impedance values of approximately 100 ohms may indicate that fenestration catheter system is in contact with a strut. Conversely, impedance values of approximately 1,000-2,000 ohms indicate that fenestration catheter system is in proper/full contact with graft cloth. Once positioned at the ostium, as described above with continuous impedance monitoring along the graft surface, fenestration catheter system 100 may be used to create a fenestration in the graft cloth while avoiding inadvertent contact with stent struts in order to properly perfuse a branch vessel without detriment to the main vessel graft supporting structure.

In addition to or in the alternative, other mechanisms may be utilized to ensure that the electrode 108 of the fenestration catheter system 100 is in full contact with cloth and not in contact with a stent strut. In one embodiment, the electrode 108 of the fenestration catheter system 100 may be utilized to create a fenestration in a graft having a longitudinal stent-free zone to facilitate forming in vivo an opening therein. For example, a tubular graft may include a proximal supported portion, an intermediate unsupported or stent-free body portion, and a distal supported portion. The proximal and distal supported portions include radially compressible stents attached thereto for supporting the ends of the graft. The intermediate body portion is solely graft material having no radial support along its length, i.e., is stent-free and unsupported, and extends between the proximal and the distal supported graft material portions. Stents support the proximal and distal ends of the graft and/or bias the proximal and distal ends of graft into conforming fixed engagement with an interior wall of a body lumen while the unsupported body portion is flexible permitting placement of the prosthesis in a highly curved anatomy, as well as reducing stresses on the graft. The length of the unsupported body portion may vary depending on the desired application and generally is about 20 mm to 40 mm. For example, when configured for cooperating with the renal arteries, the length of the unsupported body portion will be about 30 mm.

Another mechanism that may be utilized to ensure that fenestration catheter system 100 is in full contact with cloth and not in contact with a stent strut is to utilize a visualization method such as ultrasound to locate and avoid stent struts while fenestration catheter system 100 reveals the location of the branch vessel ostium. One suitable imaging catheter is the PIONEER® catheter manufactured by Medtronic Vascular, Inc. of Santa Rosa, Calif., which includes an intravascular ultrasound system (IVUS). Similar IVUS devices that may be utilized in embodiments hereof include the GALAXY™ IVUS Imaging System manufactured by Boston Scientific Corporation of Natick, Mass. and the VH™ IVUS System produced by Volcano Corporation of San Diego, Calif. In one embodiment, robotic driver interface 124 may include the capability of visualizing the stent struts as robotic control 126 is used to navigate and position fenestration catheter system at the target location.

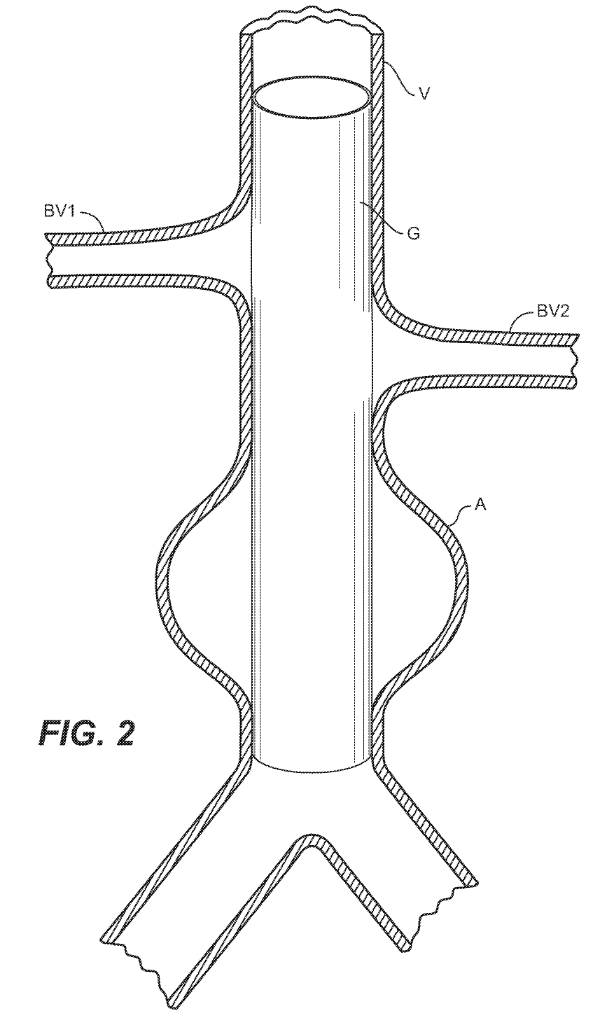

Referring now to FIGS. 2-7, a method of implanting a graft within an aneurysm and creating a fenestration in a side wall of a graft in situ utilizing real-time impedance measurements according to an embodiment hereof is described. The real-time impedance measurements aid in optimally positioning an electrode 108 with respect to an ostium of a branch vessel and the surface of the graft cloth. FIG. 2 illustrates a graft G in a deployed or expanded configuration, implanted at a treatment site within a vessel V for treating an aneurysm A. In FIG. 2, graft G is implanted within the abdominal aorta which has multiple branch vessels BV1 (the right renal artery) and BV2 (the left renal artery). The following method of creating a fenestration in a side wall of a graft in situ is described to provide perfusion to the renal arteries, but it will be understood that the method may be utilized for providing perfusion to branch vessels, such as the branch vessels of the aortic arch. Graft G is properly positioned within vessel V such that it spans aneurysm A. Methods and apparatuses for delivering graft G intravascularly are generally known in the art and may be used to place a graft delivery system within the vasculature and deliver the graft to the deployment site. For example, the graft may be guided to the deployment site using fluoroscopic imaging. Although not shown, graft G may include anchoring mechanisms such as collars having tines extending therefrom at the proximal and/or distal ends thereof to secure the prosthesis to vessel V.

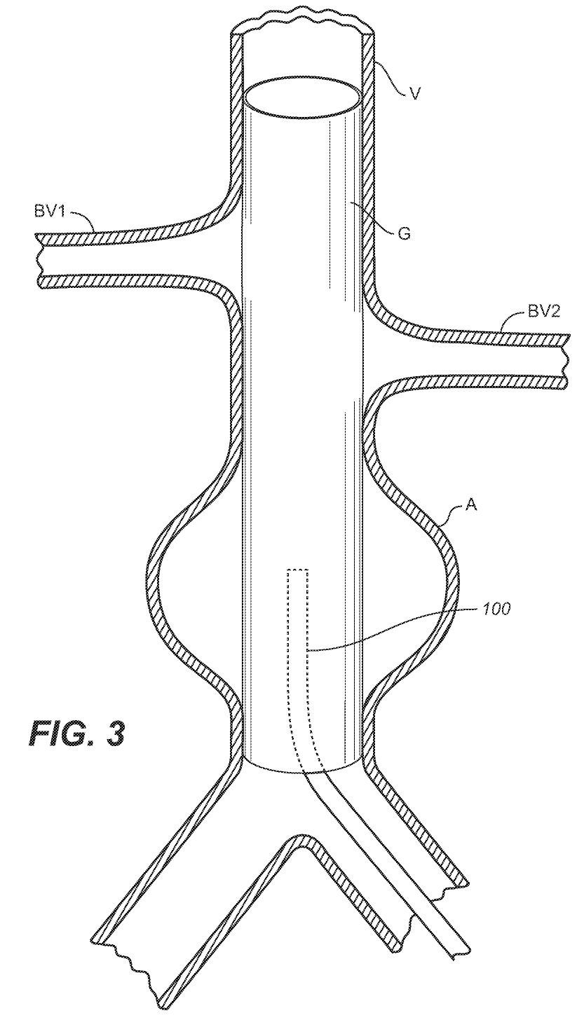

Once graft G is deployed within vessel V as discussed above, fenestration catheter system 100 may be delivered proximal to the treatment site where graft G is implanted as shown in FIG. 3. Access to the vasculature may be achieved through a branch of the femoral artery as shown, or alternatively, may be achieved through a retrograde approach such as via a carotid artery and an auxiliary artery. Methods and apparatus for delivering a catheter intravascularly are generally known in the art and may be used to place and deliver fenestration catheter system 100 within the vasculature at the target site. As described above in relation to FIGS. 1 and 9, the fenestration catheter system includes at least a tubular shaft defining at least one lumen, at least one lead extending through the at least one lumen of the tubular shaft, an electrode attached to a distal end of the lead for creating a fenestration in a wall of the endoluminal graft, a power source electrically connected to the electrode for generating an AC current, and an impedance analyzer electrically connected to the RF electrode and to a common reference electrode for continuously measuring the electrical impedance between the RF electrode and the common reference electrode.

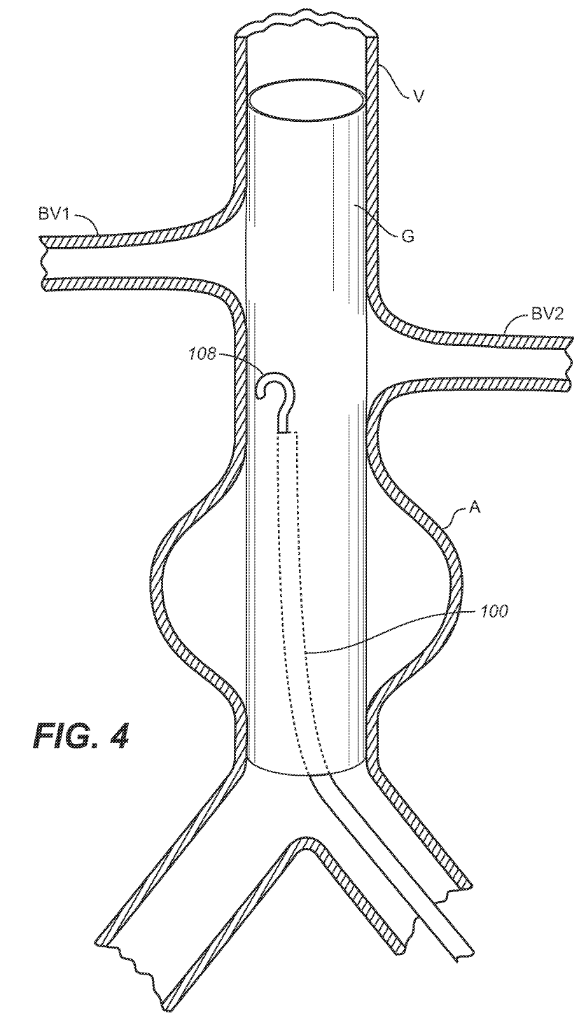

Referring now to FIG. 4, fenestration catheter system 100 is routed through the vasculature and positioned inside graft G in the vicinity of the juncture of vessel V and branch vessel BV1. As previously explained, electrode 108 of fenestration catheter system 100 may be a radiofrequency (RF) electrode formed from a self-expanding material such that it expands to at least a partial ring configuration when it is extended out of the catheter shaft. Electrode 108 is tracked along the interior surface of graft G such that fenestration catheter system 100 can continuously measure impedance. In order to measure impedance, the power source of the fenestration catheter system generates an AC current which is delivered to electrode 108 via the at least one lead extending through the catheter shaft. The impedance analyzer of fenestration catheter system 100 measures the resistance between electrode 108 and the common reference electrode (e.g., at least one skin-patch electrode), and calculates the corresponding impedance. A display mechanism may produce a virtual impedance map to show the impedance measurements or a digital readout may be obtained. In addition, a robotic steering mechanism may be utilized for manipulating fenestration catheter system 100 within the vasculature as described above with respect to FIG. 1, and the display mechanism may overlay the virtual impedance map onto a virtual 3D real time representation of the position and orientation of the system within the vasculature.

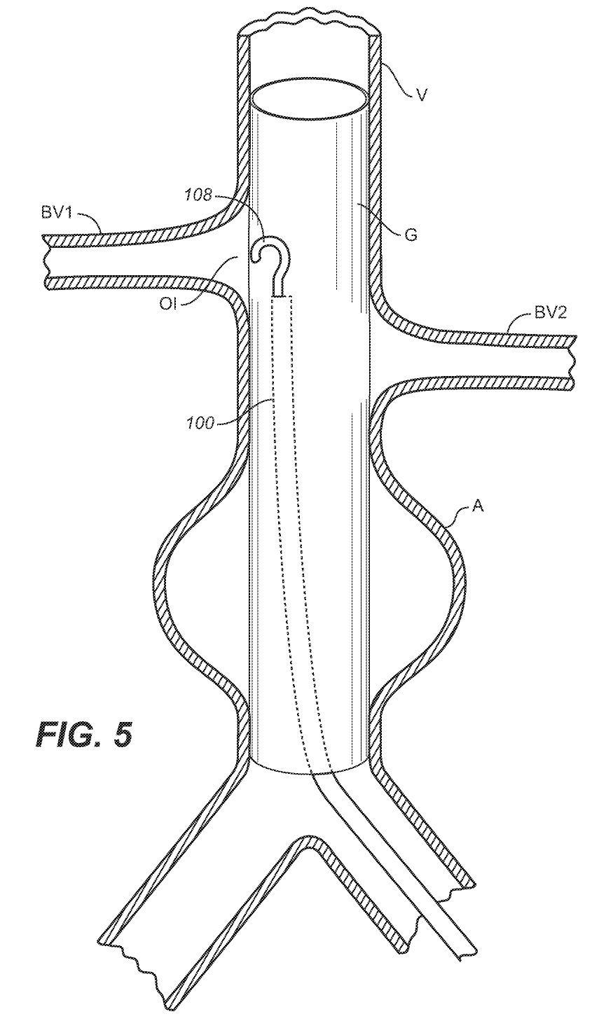

By continuously tracking electrode 108 along the interior graft surface, fenestration catheter system 100 will continuously measure relative impedance values that allow detection of the location of branch vessel ostium O1 due to the impedance differences between blood and solid tissue, i.e., the vessel wall. As previously explained above, relatively lower impedance values indicate that fenestration catheter system 100 is detecting graft material with only blood there behind and thus indicates the proper position and/or alignment with respect to the ostium O1 of target branch vessel BV1. Thus, impedance monitoring enables fenestration catheter system 100 to reveal when electrode 108 is properly aligned with the ostium O1 of target branch vessel BV1, as shown in FIG. 5.

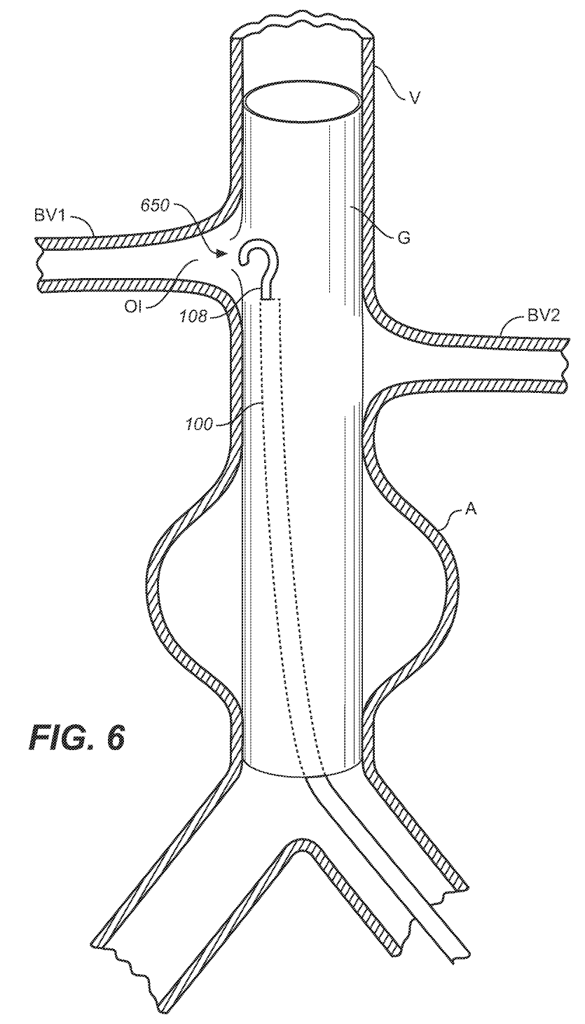

Referring now to FIG. 6, once the ostium O1 of the target branch vessel BV1 is located via monitoring of the impedance values and electrode 108 of fenestration catheter system 100 is in place adjacent a receiving area of graft G where a fenestration is to be created, electrode 108 according to its operation creates an opening or fenestration 650 in a side wall of graft G to perfuse branch vessel BV1. Opening or fenestration 650 allows blood flow through the ostium O1 and branch vessel BV1. When the branch vessel is a renal artery, for example, opening 650 provides blood flow to the kidney that the renal artery feeds.

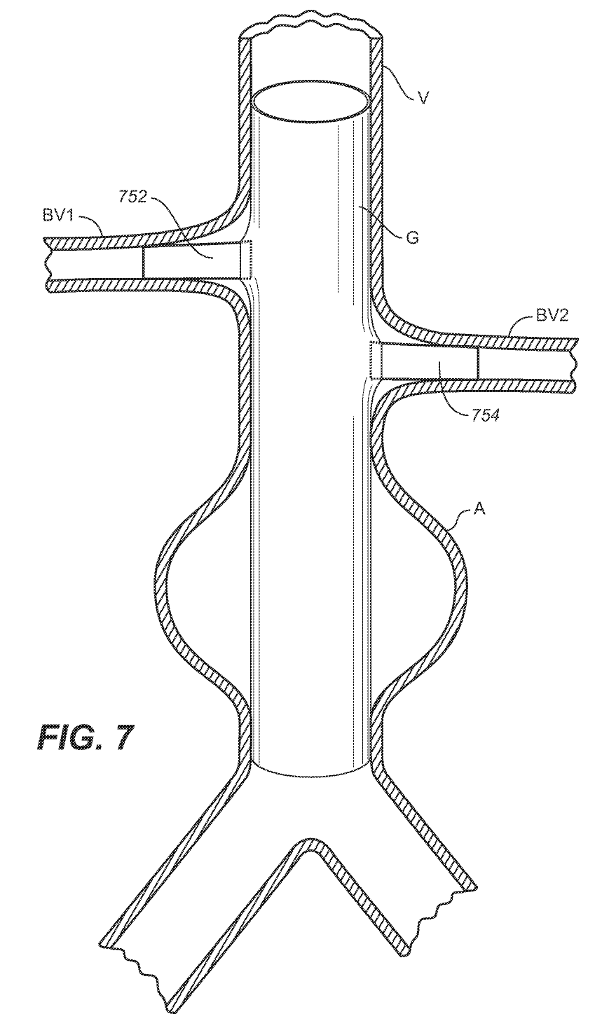

If desired, fenestration catheter device 100 may then be moved to a second branch vessel in need of perfusion, such as branch vessel BV2, and the process is repeated to create additional fenestrations in a side wall of graft G. Once fenestrations have been created in graft G as desired, fenestration catheter system 100 is removed while graft G remains expanded in the vessel against the vessel wall to provide an artificial lumen for the flow of blood. Subsequently, branch stent prostheses may be deployed via a prosthesis delivery catheter through the created fenestrations in order to support and maintain blood flow to the branch vessels. For example, FIG. 7 illustrates a branch stent graft 752 deployed within branch vessel BV1 and a branch stent graft 754 deployed within branch vessel BV2. Branch stent grafts 752, 754 seal the openings formed in graft G positioned in the main vessel V.

While various embodiments have been described above, it should be understood that they have been presented by way of illustration and example only, and not limitation. It will be apparent to persons skilled in the relevant art that various changes in form and detail can be made therein without departing from the spirit and scope of the invention. It will also be understood that each feature of each embodiment discussed herein, and of each reference cited herein, can be used in combination with the features of any other embodiment. All patents and publications discussed herein are incorporated by reference herein in their entirety.

What is claimed is:

1. An intravascular system that facilitates fenestration of an endoluminal graft in situ, the system comprising:
a tubular shaft defining at least one lumen;
at least one lead extending through the at least one lumen of the tubular shaft;
an electrode attached to a distal end of the at least one lead, wherein the electrode is capable of creating a fenestration in a wall of the endoluminal graft;
a power source electrically connected via the at least one lead to the electrode, wherein the power source generates an AC current; and
an impedance analyzer electrically connected to the electrode and to a common reference electrode, wherein the impedance analyzer measures the electrical impedance between the electrode and the common reference electrode at positions relative to a reference location to identify an optimal location for creating a fenestration in a wall of the endoluminal graft.

2. The system of claim 1, wherein the impedance analyzer measures impedance associated with graft material having only blood there behind and impedance associated with graft material having solid tissue there behind.

3. The system of claim 1, wherein the impedance analyzer measures impedance associated with cloth of the endoluminal graft and impedance associated with a stent strut of the endoluminal graft.

4. The system of claim 1, wherein the electrode is a radiofrequency (RF) electrode of at least a partial ring shape.

5. The system of claim 4, wherein the electrode is formed from a self-expanding material.

6. The system of claim 1, wherein the common reference electrode is a skin patch electrode.

7. The system of claim 1, further comprising:
a robotic steering interface for deflecting and rotating the system in all directions; and
a display for producing a virtual 3D real time representation of the position and orientation of the system within the vasculature.

8. The system of claim 1, further comprising:
a display for producing a virtual impedance map of impedance measurements.

9. The system of claim 8, wherein the display overlays the virtual impedance map onto a virtual 3D real time representation of a position and orientation of the system within the vasculature.

10. An intravascular system that facilitates fenestration of an endoluminal graft in situ, the system comprising:
a tubular shaft defining at least one lumen;
a radiofrequency (RF) electrode slidably disposed within the tubular shaft, wherein the RF electrode is capable of creating a fenestration in a wall of the endoluminal graft;
a power source electrically connected to the RF electrode for generating an AC current;
an impedance analyzer electrically connected to the RF electrode and to a common reference electrode, wherein the impedance analyzer measures the electrical impedance between the RF electrode and the common reference skin-patch electrode to provide an optimal location for creating a fenestration in a wall of the endoluminal graft;
a robotic steering interface for deflecting and rotating the system within the vasculature; and
a display for producing a virtual 3D real time representation of the position and orientation of the system within the vasculature and for producing a virtual impedance map of impedance measurements, wherein the display overlays the virtual impedance map onto the virtual 3D real time representation of the position and orientation of the system within the vasculature.

11. The system of claim 10, wherein the impedance analyzer measures impedance associated with graft material having only blood there behind and impedance associated with graft material having solid tissue there behind.

12. The system of claim 10, wherein the impedance analyzer measures impedance associated with cloth of the endoluminal graft and impedance associated with a stent strut of the endoluminal graft.

13. A method of forming a fenestration in a wall of an endoluminal graft in situ, the method comprising the steps of:
advancing a fenestration catheter system within an endoluminal graft in a first passageway in a human body, wherein the graft obscures an ostium of a second passageway that branches from the first passageway, wherein the fenestration catheter system includes a tubular shaft defining at least one lumen, a radiofrequency (RF) electrode disposed within the tubular shaft, a power source for generating an AC current electrically connected to the RF electrode, and an impedance analyzer electrically connected to the RF electrode and to a common reference electrode,
continuously measuring the electrical impedance between the RF electrode and the common reference electrode via the impedance analyzer as the location of the electrode is changed on the surface of the graft material to determine an optimal location for creating a fenestration in a wall of the endoluminal graft; and
creating a fenestration in the wall of the endoluminal graft at the optimal location via the RF electrode, wherein the fenestration allows for blood flow through the ostium of the second passageway.

14. The method of claim 13, wherein the optimal location occurs when the RF electrode is aligned with a branch vessel ostium and is determined via a comparison of impedance measurements associated with graft material having only blood there behind and impedance measurements associated with graft material having solid tissue there behind.

15. The method of claim 13, wherein the optimal location is determined via a comparison of impedance measurements associated with the RF electrode in contact with cloth of the endoluminal graft and impedance measurements associated the RF electrode in contact with a stent strut of the endoluminal graft.

16. The method of claim 13, wherein the RF electrode has at least a partial ring shape.

17. The method of claim 13, wherein the common reference electrode is a skin patch electrode.

18. The method of claim 13, wherein the step of advancing a fenestration catheter system within the endoluminal graft includes robotically steering, deflecting, and rotating the fenestration catheter system.

19. The method of claim 13, further comprising:
displaying a virtual impedance map including impedance measurements.

20. The system of claim 19, wherein the step of displaying includes overlaying the virtual impedance map onto a virtual 3D real time representation of the position and orientation of the system within the vasculature.
